(19) United States
(12) Patent Application Publication
(10) Pub. No.: US 2012/0078365 A1
(43) Pub. Date: Mar. 29, 2012

(54) METHOD FOR RECONSTRUCTION AND AUGMENTATION OF THE BREAST

(76) Inventors: Dominique Erni, Kussnacht (CH); Jan A. Plock, Kiesen (CH)

(21) Appl. No.: 12/890,078

(22) Filed: Sep. 24, 2010

(51) Int. Cl. A61F 2/12

(52) U.S. Cl. 623/8

(57) ABSTRACT

The invention provides a method for manufacturing a breast prosthesis, which method comprises the determination of existing contours of a breast of an individual in an upright position using magnetic resonance imaging (MRI) or ultrasound (US), the planning of a moulage or virtual moulage using a computer-assisted modeling software, with or without a 3D visualization of a result to be expected, and the manufacturing of a customized breast prosthesis based on said moulage or said virtual moulage. The invention also provides for methods of breast reconstruction and/or augmentation.

### FIELD OF THE INVENTION

The present invention generally relates to a method for breast reconstruction and augmentation.

### BACKGROUND

Breast cancer is the most common tumor in women. In Western countries this pathology concerns about one woman out of seven. Thanks to improved early detection and enhanced treatment options, the survival rate increased significantly over the past years, which in turn increased the demands for an aesthetic result. Two-thirds of all cancers can be treated by breast-conserving measures, whereas in one third of the cases a mastectomy is unavoidable.

Among these latter cases, one third ask for the surgical restoration of the breast. Such a restoration can be done either by using autologous tissue or by implantation of silicone prostheses (expander prostheses, followed by permanent implants), or combinations of both.

A method propagated by the inventor consists in expanding in a first-stage the remaining breast skin using a prosthetic expander and to thereafter replace the prosthesis by transplantation of autologous tissue.

Furthermore, women may ask for augmentation of one or both of their breasts for various reasons, such as to correct different size of the breasts, to correct changes of the breasts after pregnancy, lactation, or aging. Moreover, many patients having reported distress and endured teasing about their appearance may gain improved confidence and self-esteem by augmentation mammoplasty. Finally, breast augmentation may be performed for cosmetic reasons only.

Although the goal of breast reconstruction and augmentation is to recreate symmetric natural-appearing breasts while preserving patient safety and quality of life, many techniques exist for breast reconstruction, but rarely do they produce true symmetry with the contralateral breast or a shape which actually corresponds to what the individual considers natural and acceptable as a part of her own.

In fact, current breast prostheses (i.e. mammary prosthetic implants) must be selected among prefabricated (off-the-shelf) prostheses from different manufacturers with slightly varying assortments, which per se gives rise to the following drawbacks:

in case of partial removal of breast tissue, there are no implants with an amorphous structure similar to that of the actually removed tissue,

in case of a mastectomy, the off-the-shelf implants usually are too dissimilar to the contralateral breast, both as an expander or as a permanent implant,

in case of breast augmentation, the results after implantation of off-the-shelf implants are too often unsatisfactory to the patients themselves as the shape of the augmented breast does not mirror the wide variations in natural breast shapes and therefore does often not correspond to the particular expectations to the actual shape and individual aesthetical feelings, which in turn is largely due to the limited available implant shapes, but also the individual variations in inner breast and chest structures.

### SUMMARY

It is a general object of the invention to provide a method to overcome at least some of the drawbacks of known methods.

In particular, a first object of the invention is to provide a method for manufacturing a breast prosthesis or prosthetic implant which after implantation essentially preserves the breast's original shape and size and/or which provides a result essentially matching the mirrored shape and size of the (non affected) contralateral breast.

To achieve this object, the present invention proposes a method for manufacturing a breast prosthesis, which method comprises the following steps:

a. determination of existing contours of a breast of an individual in an upright position using magnetic resonance imaging (MRI) or ultrasound (US),

b. planning of a moulage or a virtual moulage using a computer-assisted modeling software, with or without a 3D visualization of a result to be expected.

c. manufacturing of a customized breast prosthesis based on said moulage or said virtual moulage.

In the context of the present invention, the planning of a “moulage” using a computer-assisted modeling software may comprise the step of manufacturing a real physical moulage or not, in which latter case the planned moulage may also be referred to as “virtual moulage” or “virtual implant” herein.

In a further, preferred aspect, the method comprises in step b the planning of said physical or virtual moulage by taking into account the individual's demands regarding shape and/or size of said moulage, respectively of the reconstructed breast.

In a further aspect, the method comprises in step c the manufacturing of a customized expander prosthesis, a customized permanent prosthesis or both.

Further objects of the invention relate to breast reconstruction and/or augmentation. Breast reconstruction is generally indicated in cases of mammary cancer, but may also occur in cases of congenital dysfigurement, asymmetries, injury or accident. In particular in the case of mammary cancer, it must be assured that the tumorous tissue is entirely removed to prevent relapse of the tumor. Hence, surgical resection of a diagnosed tumor generally comprises, as a precautionary measure, the resection of the tumorous tissue itself and of resection margins in the surrounding healthy tissue. The extent of the margins generally depends on various factors including breast size, number of lesions, biologic aggressiveness of a breast cancer, etc.

In the context of the present invention, the expressions “tissue to be resected” or “resected tissue” (as opposed to remaining tissue) thus refer to both the tumorous tissue and surrounding healthy tissue which needs to be or has been removed.

A second object of the invention is to provide a method of reconstructive surgery of a breast with a diagnosed tumor of the mammary tissue which after surgery essentially preserves the breast's original shape and size.

Hence in a further aspect, the invention provides a method for breast reconstruction of a tumorous breast of an individual, wherein said tumorous breast comprises tissue to be resected and remaining tissue, which method comprises the following steps:

pre-surgical determination of existing contours of a breast of said individual in an upright position using magnetic resonance imaging (MRI) or ultrasound (US),

determination of size, shape and location of the tissue to be resected, i.e. tumorous tissue including required resection margins of the surrounding healthy tissue, within said tumorous breast,

planning of a moulage or virtual moulage corresponding to said size and shape of the tissue to be resected using a computer-assisted modeling software, with or without a 3D visualization of a result to be expected, manufacturing of a customized breast prosthesis corresponding to said moulage or said virtual moulage,

surgical resection of said tissue to be resected and

implantation of said customized breast prosthesis at the location of said resected tissue.

A further advantage of the above method is the fact that it can also be performed bilaterally.

A third object of the invention is to provide a method of reconstructive surgery of a breast with a diagnosed tumor of the mammary tissue which after surgery provides a result essentially matching the mirrored shape and size of the (non affected) contralateral breast.

Hence in a still further aspect, the invention provides a method for breast reconstruction of a tumorous breast of an individual having a tumorous and a healthy breast, wherein said tumorous breast comprises tissue to be resected and remaining tissue, which method comprises the following steps:

pre-surgical determination of existing contours of the healthy breast of said individual in an upright position using magnetic resonance imaging (MRI) or ultrasound (US),

determination of size and shape of said healthy breast,

determination of the remaining tissue in the tumorous breast,

projection of a mirror image of the contralateral, healthy breast contours to the tumorous breast,

planning of a moulage or virtual moulage corresponding to the substraction of the said remaining tissue on the tumorous breast from said projected mirror image using a computer-assisted modeling software, with or without a 3D visualization of a result to be expected,

manufacturing of a customized breast prosthesis corresponding to said moulage or virtual moulage,

surgical resection of said tissue to be resected from said tumorous breast and

implantation of said customized breast prosthesis at a location of said resected tissue.

This method is particularly advantageous in cases where the shape and/or size of the affected, tumorous breast is already affected by the cancer.

A fourth object of the invention is to provide a method of reconstructive surgery of a mastectomy which after reconstructive surgery provides a result essentially matching the mirrored shape and size of the (non affected) contralateral breast.

Hence in a still further aspect, the invention provides a method for breast reconstruction of a breast of an individual after monolateral mastectomy and having a contralateral healthy breast, which method comprises the following steps:

pre-surgical determination of existing contours of said healthy breast of said individual in an upright position using magnetic resonance imaging (MRI) or ultrasound (US),

determination of size and shape of said healthy breast,

projection of a mirror image of the contralateral, healthy breast contours to the mastectomy site,

planning of a moulage or virtual moulage corresponding to the substraction of remaining tissue on the mastectomy site from said projected mirror image using a computer-assisted modeling software, with or without a 3D visualization of a result to be expected,

manufacturing of a customized breast prosthesis corresponding to said moulage or virtual moulage,

implantation of said customized breast prosthesis at an initial location of said mastectomized/amputated breast.

In the context of the present invention, a customized breast prosthesis or customized mammary prosthetic implant may be an expander prosthesis or a permanent prosthesis, such as saline-filled and silicone-gel-filled implants.

If in the above-described methods the customized breast prosthesis is an expander prosthesis, the methods further comprise the steps of either or both of:

removal of the expander prosthesis and substitution with autologous tissue, or

manufacturing of a customized permanent breast prosthesis corresponding to said moulage or said virtual moulage,

surgical removal of said customized expander prosthesis after expansion of said breast and

implantation of said customized permanent breast prosthesis at a location of said expander prosthesis.

A fifth object of the invention is to provide a method for breast augmentation which after surgery provides a result more closely matching the expectations of the patient with regard to the breast's shape and size, in particular by taking into account the individual aesthetical preferences of the patient.

Hence in a still further aspect, the invention provides a method for breast augmentation in an individual, which method comprises the following steps:

pre-surgical determination of existing contours of both breasts of said individual in an upright position using magnetic resonance imaging (MRI) or ultrasound (US),

planning of moulages or virtual moulages taking into account the individual's demands using a computer assisted modeling software with a 3D visualization of a result to be expected,

manufacturing of customized breast prostheses corresponding to said moulages or said virtual moulages,

implantation of said customized breast prostheses.

The main advantage of the above methods is that they can be used for a variety of breast shapes and sizes, and that the aspect of left-right mirroring is respected by the use of implants that are asymmetrical in shape (symmetry between left and right implant).

A further major advantage is the enhanced acceptance of the implanted breast and thus a reduced emotional trauma due to a better correlation between the patient's expectations and the actual result.

Another advantage of such customized implants is that they are less likely to rotate after implantation due to their irregular, asymmetrical shape.

A still further advantage provided by the above methods is the fact that minor corrective adjustments to the original shape and contours are easily performable during the planning of the moulage or the virtual moulage. In case of bilateral surgery where there is no need for symmetry with a non affected contralateral breast, more important corrective adjustments are conceivable without departing from the above described methods.

In the present context, the expression “breast reconstruction” actually generally refers to both breast reconstruction and breast augmentation if not otherwise stated.

Further details and advantages of the present invention will be apparent from the following detailed description of several not limiting embodiments.

### DETAILED DESCRIPTION

An important step in the above-described methods is the determination of the existing contours of a individual's breasts. In the present context the term “contours” does not only refer to the external shape of the breast, but also to the internal tissue structure, including the mammary gland, the chest wall, the pectoralis muscles, the nipple, the areola, the fatty tissue and the skin.

A major advantage of both magnetic resonance imaging (MRI) and ultrasound (US) vs. computer tomography (CT) is the fact that the body of the individual (patient) is not exposed to X-rays. These techniques furthermore allow to safely identify and locate the tissue to be removed or resected (such as tumorous tissue). Moreover, upright MRI and upright US allow taking into account gravitational forces on the shape of the breast. Indeed, as the examination is conventionally conducted with the patient being in a horizontal (recumbent) position, MRI is not used in reconstructive surgery, because the reconstructed breast must actually be shaped for the upright position. Currently, upright MRIs are performed only in relation with orthopedic problems, in particular in case of pain associated with the spinal column. One example of a MRI device appropriate for use in the present invention is the Fonar Upright® MRI (FONAR Corporation, Melville, N.Y. 11747). Examples of ultrasound devices suitable for use in the present invention are commercialized by Sonoace GmbH, Marl, Germany or Siemens Medical Solutions, Malvern, Pa.

Hence, contrary to surface scanners, upright MRI and US allow to identify deeper structures and to record the actual thickness of the soft tissue, both of which have significant impact on the final result.

The use of computer-assisted modeling software allows the shaping of the moulage or the virtual implant while being able to visualize the corresponding virtual result in a three dimensional mode on screen. Furthermore, the virtual result can be easily altered, amended and changed without requiring particular computer skills. Even more importantly, the patient may be involved in the planning of the moulage or the virtual implant, which enhances both the self-esteem and the acceptance of the prostheses after implantation and is the prerequisite to design the implant shape exactly according to the patient's desire.

As already mentioned above, there are two main types of permanent breast implants: saline-filled and silicone-gel-filled implants. Saline implants generally have a silicone elastomer shell filled with sterile saline liquid, whereas silicone gel implants have a silicone shell filled with a viscous silicone gel.

Expander prostheses or breast tissue expanders are temporary breast implants used during staged breast reconstruction procedures.

The term “customized” in relation with breast prostheses as used herein refers to the fact that the prosthetic implants have a shape, configuration and size specially adapted to the individual's needs, such as in particular in view of her pathological, anatomical and emotional conditions. The shape and configuration of a customized breast prosthesis generally significantly differ from those of off-the-shelf implants. Indeed, compared to conventional prostheses, the present methods take into account contralateral (left-right) differences. In addition, anatomical particularities, such as geometry of the chest, position of the nipple, local presence or absence of mammary tissue, etc. Finally the customized prosthesis does not only provide a result which correspond to the patient's expectations with regard to size and rudimentary shape (round, tear shaped), but as a whole. The methods of the present invention exceed the potential of know asymmetrical implants (e.g. from Poly Implant Prothese (PIP) company, France), which are produced off-shelf on a non-individual basis and are based on imaging in a recumbent position, thus not respecting gravitational forces and individual design.

Breast implants for reconstruction or augmentation may be placed via various types of incisions, such as inframammary, periareolar or transaxillary incisions.

In the inframammary approach, an incision is placed below the breast in the infra-mammary fold. This incision is the most common approach and affords maximum access for precise dissection and placement of an implant. It is often the preferred technique for silicone gel implants due to the longer incisions required.

A periareolar incision is placed along the areolar border and provides an optimal approach when adjustments to the infra-mammary fold position or mastopexy procedures are planned. The incision is generally placed around the inferior half, or the medial half of the areola's circumference.

A transaxillary incision is placed in the armpit and the dissection tunnels medially. This approach allows implants to be placed with no visible scars on the breast.

Furthermore, the implants may be placed directly under the skin, gland and fatty tissue, or underneath the muscle tissue and/or its fascia entirely or in parts.

Further usable techniques employ transumbilical incision or transabdominoplasty.

A “tumor” or a “tumorous tissue” in the present context is to be understood as being any neoplasm or neoplastic tissue due to abnormal proliferation of cells. Said neoplasms may be benign, pre-malignant or malignant. Furthermore, in the present context, these expressions also refer to other breast pathologies associated with tissue alterations, such as, but not limited to, fibrocystic breast changes. In general, these expressions broadly refer to any abnormal or non healthy tissue or aggregate, resp. to any so-called breast lump, such as, but not limited to, cysts, sebaceous cysts, abscesses, adenomas, fibroadenomas, etc.

We claim:

1. A method for manufacturing a breast prosthesis, which method comprises:
a. determination of existing contours of a breast of an individual in an upright position using magnetic resonance imaging (MRI) or ultrasound (US),
b. planning of a moulage using a computer-assisted modeling software,
c. manufacturing of a customized breast prosthesis based on said moulage planned in step b.

2. The method as claimed in claim 1, wherein planning of said moulage takes into account the individual's demands regarding shape and/or size of said moulage.

3. The method as claimed in claim 1, wherein the planning of said moulage comprises a 3D visualization of a result to be expected.

4. The method as claimed in claim 1, wherein manufacturing the customized breast prosthesis includes manufacturing a customized expander prosthesis, a customized permanent prosthesis or both.

5. The method as claimed in claim 2, wherein manufacturing the customized breast prosthesis includes manufacturing a customized expander prosthesis, a customized permanent prosthesis or both.

6. A method for breast reconstruction of a tumorous breast of an individual, wherein said tumorous breast comprises tissue to be resected, which method comprises:
pre-surgical determination of existing contours of said tumorous breast of said individual in an upright position using magnetic resonance imaging (MRI) or ultrasound (US);
determination of size, shape and location of the tissue to be resected within said tumorous breast;
planning of a moulage corresponding to said size and shape of said tissue to be resected using a computer-assisted modeling software;
manufacturing of a customized breast prosthesis corresponding to said moulage;
surgical resection of said tissue to be resected; and
implantation of said customized breast prosthesis at the location of said resected tissue.

7. The method as claimed in claim 6, wherein the planning of said moulage comprises a 3D visualization of a result to be expected.

8. A method for breast reconstruction of a tumorous breast of an individual having a contralateral healthy breast, wherein said tumorous breast comprises tissue to be resected and remaining tissue, which method comprises:
pre-surgical determination of existing contours of a healthy breast of said individual in an upright position using magnetic resonance imaging (MRI) or ultrasound (US);
determination of size and shape of said healthy breast;
determination of the remaining tissue in the tumorous breast;
projection of a mirror image of the contralateral healthy breast contours to the tumorous breast;
planning of a moulage corresponding to the subtraction of the said remaining tissue of the tumorous breast from said projected mirror image using a computer-assisted modeling software;
manufacturing of a customized breast prosthesis corresponding to said moulage;
surgical resection of said tissue to be resected from said tumorous breast; and
implantation of said customized breast prosthesis at a location of said resected tissue.

9. A method for breast reconstruction of a breast of an individual after monolateral mastectomy and having a contralateral healthy breast, which method comprises:
pre-surgical determination of existing contours of a healthy breast of said individual in an upright position using magnetic resonance imaging (MRI) or ultrasound (US);
determination of size and shape of said healthy breast;
projection of a minor image of contours of the contralateral healthy breast to the mastectomy site;
planning of a moulage or virtual implant corresponding to the subtraction of the remaining tissue on the mastectomy site from said projected mirror image using a computer-assisted modeling software;
manufacturing of a customized breast prosthesis corresponding to said moulage; and
implantation of said customized breast prosthesis at an initial location of said mastectomized breast.

10. The method as claimed in claim 9, wherein the planning of said moulage comprises a 3D visualization of a result to be expected.

11. The method as claimed in claim 9, wherein said customized breast prosthesis is a customized expander prosthesis.

12. The method as claimed in claim 11, further comprising removal of the expander prosthesis and substitution with autologous tissue.

13. The method as claimed in claim 12, further comprising:
manufacturing of a customized permanent breast prosthesis corresponding to said moulage,
surgical removal of said customized expander prosthesis after expansion of said breast and
implantation of said customized permanent breast prosthesis at a location of said expander prosthesis.

14. A method for breast augmentation in an individual, which method comprises:
pre-surgical determination of existing contours of both breasts of said individual in an upright position using magnetic resonance imaging (MRI) or ultrasound (US),
planning of moulages taking into account the individual's demands using a computer-assisted modeling software,
manufacturing of customized breast prostheses corresponding to said moulages,
implantation of said customized breast prostheses.

15. The method as claimed in claim 14, wherein the planning of said moulages comprises a 3D visualization of a result to be expected.
